(19) United States
(12) Patent Application Publication
(10) Pub. No.: US 2007/0088157 A1
(43) Pub. Date: Apr. 19, 2007

(54) Preparation of glucosamine

(76) Inventor: John Clark Hubbs, Kingsport, TN (US)

Correspondence Address: Brett L. Nelson;Eastman Chemical Company, P.O. Box 511, Kingsport, TN, 37662-5075, US

(21) Appl. No.: 11/293,757

(22) Filed: Dec. 2, 2005

Related U.S. Application Data
(60) Provisional application No. 60/727,481, filed on Oct. 17, 2005.

(51) Int. Cl. C07H 5/04

(52) U.S. Cl. 536/55.3

(57) ABSTRACT

Disclosed is a process for the preparation of a glucosamine acid addition salt from fructose and ammonia or an ammonia source such as an ammonium compound by contacting fructose and ammonia or an ammonia source in the presence of (i) a solvent comprising about 25 to 100 weight percent water and 75 to 0 weight percent of an inert, organic, water-miscible solvent at a ^{W}_{W}pH or ^{S}_{W}pH of about 1 to 6; or (ii) a solvent comprising about 75 to 100 weight percent of an inert, organic, water-miscible solvent and 0 to 25 weight percent water at a ^{W}_{W}pH or ^{S}_{W}pH of about 1 to 10. A mannosamine acid addition salt also is produced as a co-product of the process.

### CROSS-REFERENCE TO RELATED APPLICATIONS

This application claims priority to U.S. Provisional Application Ser. No. 60/727,481 filed Oct. 17, 2005, the disclosure of which is incorporated herein by reference in its entirety.

### FIELD OF THE INVENTION

This invention pertains to a process for the preparation of glucosamine from fructose and ammonia or an ammonia source such as an ammonium compound. More specifically, this invention pertains to the preparation of glucosamine by contacting fructose and ammonia or an ammonia source in the presence of water at a pH of 6 or less or in the presence of an inert, organic, water-miscible solvent at a ^{S}_{W}pH of about 1 to 10. Mannosamine also is produced as a co-product of the process.

### BACKGROUND OF THE INVENTION

Glucosamine is an amino sugar found in many naturally occurring polysaccharides. Glucosamine has found increasing use in the treatment of osteoarthritis as has been recently reviewed by Clegg et. al. (D. O. Clegg, C. G. Jackson in Encyclopedia of Dietary Supplements, Marcel Dekker Publisher, 2005, page 279). Most glucosamine is prepared commercially by acid hydrolysis of chitin [poly-beta-(1-4)-N-acetyl-D-glucosamine] which is a major component of the shells of crustaceans such as crabs and shrimp. Because it is derived from shellfish hydrolyzates, persons with shellfish allergies may need to avoid exposure or use with caution. Furthermore, the availability of the shellfish raw material is becoming increasingly limited. More recently, methods for the production of glucosamine by microbial fermentation have been disclosed. U.S. Pat. No. 6,372,457 discloses fermentation microorganisms that have been genetically modified in a pathway related to glucosamine or glucosamine-6-phosphate in order to, produce high levels of glucosamine. This fermentation process suffers from relatively long fermentation times and relatively low yields partially due to the need to build up cell mass from the sugar feed. There remains a need to produce glucosamine from a shellfish-free source using abundant and inexpensive starting materials.

Heyns reports in U.S. Pat. No. 2,884,411 the preparation of D-(+)-glucosamine and salts of glucosamine such as glucosamine hydrochloride from D-fructose in either liquid ammonia or strongly alkaline aqueous ammonium hydroxide. Heyns notes that the instability of glucosamine under the reaction conditions requires immediate neutralization and isolation. Heyns et al., Berichte; 90, 2039, (1957), disclose yields ranging from traces of glucosamine to up to approximately 30% from fructose and ammonia based on isolated solids, e.g., Example 1 of U.S. Pat. No. 2,884,411, although their experimental data does not reveal the purity of those solids relying. primarily on the weight of isolated solids and optical rotation. The decomposition of glucosamine under the reaction conditions renders the process of Heyns very difficult to practice. Heyns and Meinecke, Berichte; 86, 1453 (1953), also report yields of glucosamine of up to approximately 30% based on the colorimetric method of Schloss, Analytical Chemistry, 23, 1321 (1951), which is reported by Schloss to have variable results as a function of pH, incubation temperature and incubation time. Teglia et. al., Anales de la Asociacion Quimica Argentina, 61, 153 (1973), discuss the production of glucosamine from fructose in strongly alkaline. ammonia in methanol and determine the identity of the product by paper chromatography. No yield is given. The reaction of high fructose corn syrup and ammonium hydroxide at temperatures between 25 and approximately 50° C. under conditions very similar to those of Heyns to produce glucosamine and lesser amounts of mannosamine and galactosamine in yields of approximately 8% (based on the high fructose corn syrup feed) is disclosed in U.S. Pat. Nos. 6,440,223. US 6,440,223 does not disclose a method to isolate the glucosamine, reports molar ratios of glucosamine/mannosamine of between 1/1 and 4/1, and the decomposition of glucosamine (as noted by others, vide infra) under the reaction conditions employed makes the patented process difficult to practice.

Heyns and Koch report in Z. Physiol. Chem. 296, 121 (1954) the oxidative degradation of glucosamine at alkaline pH in the presence of air in aqueous solutions at 38° C. They also report the anaerobic decomposition of glucosamine at 38° C. at slightly acidic pH and alkaline pH in aqueous solution. Heyns and Koch suggest an acceleration of the rate of decomposition as a result of phosphate buffer. The suggested decomposition products include fructosazine, arabinose, hydroxyl-methylfurfural, glyceraldehyde and ammonia which were primarily characterized by paper chromatography. Zimmerman reports in Archives of Biochemistry and Biophysics, 82, 266 (1959) the instability of glucosamine above a pH of 5 at temperatures as low as 30° C. when using an indirect method of consumption of alkali. Lea et al., report in Nature, 169, 1097 (1952) the decomposition of glucosamine from solids of glucosamine and casein which had been freeze dried at a pH above 6.3, e.g., approximately 50% decomposition in 2-5 days at 80% relative humidity at 38° C.). Solutions freeze dried under a pH of 6.3 were reported to be stable. Lea, et. al. conclude that glucosamine is stable when protonated and state that glucosamine is more than 95% protonated under a pH of 6.3. Eitelman et al, report in Carbohydrate Research, 77, 213 (1979) that glucosamine is stable for 24 hours at 27° C. in 8-16 M sodium hydroxide but undergoes “considerable” decomposition in 0.75 hours in 8 M sodium hydroxide at 60° C. based on NMR analysis. Shao et al. report in Journal of Pharmaceutical and Biomedical Analysis, 35, 625 (2004) 10-20% degradation of glucosamine in 1 day at room temperature in aqueous solution at a pH of 11. Taha reports in Journal of the Chemical Society, Abstracts (1961), 2468-72 that the optical rotation of glucosamine disappears in 7 days at room temperature in aqueous ammonia.

### BRIEF DESCRIPTION OF THE INVENTION

I have discovered that glucosamine may be prepared from fructose and ammonia or an ammonium compound under certain conditions. The present invention provides a process for the preparation of a glucosamine acid addition salt which comprises contacting fructose with ammonia, an ammonium compound or a mixture thereof in the presence of:
(i) a solvent comprising about 25 to 100 weight percent water and 75 to 0 weight percent of an inert, organic, water-miscible solvent at a ^{W}_{W}pH or ^{S}_{W}PH of about 1 to 6; or
(ii) a solvent comprising about 75 to 100 weight percent of an inert, organic, water-miscible solvent and 0 to 25 weight percent water at ^{S}_{W}pH of about 1 to 10. It has been found that acid addition salts of glucosamine are stable or more stable in an acidic and/or substantially anhydrous environment and may be isolated with little or no decomposition. While crystalline fructose is a preferred substrate for reaction with ammonia or ammonium salts, high fructose syrup may be used as a cheap source containing fructose. Preferred are high fructose syrups containing greater than 40% fructose. Even more preferred are those syrups containing more than 50% fructose. Especially preferred as fructose feeds are purified streams of high fructose syrup containing more than 80% fructose, e.g., aqueous fructose solutions containing about 80 to 95 weight percent fructose. Such streams typically result from the ion exchange chromatography used to purify high fructose corn syrup.

### DETAILED DESCRIPTION

According to the present invention, fructose is contacted with ammonia, an ammonium compound or a mixture thereof in the presence of a solvent comprising (i) about 25 to 100 weight percent water and 75 to 0 weight percent of an inert, organic, water-miscible solvent at ^{W}_{W}pH or ^{S}_{W}pH of about 1 to 6; or (ii) about 75 to 100 weight percent of an inert, organic, water-miscible solvent and 0 to 25 weight percent water at ^{s}_{w}pH of about 1 to 10 to produce glucosamine. The weight percentages of the components of the solvent, i.e., the weight percentages of water and inert, organic, water-miscible solvent, are based on the total weight of the solvent exclusive of the weight of dissolved or suspended reactants, products and/or buffers. For example, for a solvent composition containing 25% water and 75% of an inert, organic, water-miscible solvent, the weight percentages are based only on the total weight of water and inert, organic, water-miscible solvent. The process typically is operated using greater than 7, preferably greater than 20, weight percent dissolved or suspended solids based on the total weight of the reaction mixture. As used herein glucosamine refers to D-(+)-glucosamine and all of the acid addition salts of glucosamine which include the commercial salt forms of glucosamine hydrochloride, glucosamine sulfate, glucosamine sulfate.2KCl, and glucosamine sulfate.2NaCl. The fructose employed in my novel process may be crystalline fructose or it may be provided in a more economical water solution, i.e., a syrup, such as high fructose syrup. Preferred aqueous solutions comprise high fructose syrups containing greater than 40 weight percent fructose, more preferably high fructose syrups containing more than 50 weight percent fructose and most preferably purified streams of high fructose syrup containing more than 80 weight percent fructose. Such streams may be obtained by the ion exchange chromatography used to purify high fructose corn syrup. The fructose concentration in the process solvent may range from about 1 to 40 weight percent based on the total weight of the reaction mixture. Concentration of about 5 to 30 weight percent (same basis) are more typical.

As used herein to describe nonaqueous pH, e.g., pH measured in non-aqueous or partially aqueous conditions of between 0 and 99% water, nonaqueous pH refers to ^{S}_{W}pH as defined by T. Mussini et. al. (T. Mussini, A. K. Covington, P. Longhi, S. Rondinini, in Criteria for Standardization of pH Measurements in Organic Solvents and Water+Organic Solvent Mixtures of Moderate to High Permittivities, Pure and Appl. Chem., 57, 865 (1985) and by Helmuth Galster et. al. (Helmuth Glaster, pH Measurement: Fundamentals, Methods, Application, Instrumentation, VCH Pub (1981) wherein the nonaqueous pH measurement is made with a pH meter using an electrode which is calibrated in water but from which the reading is obtained in the non-aqueous or partially aqueous solvent. Such nonaqueous pH measurements may be made in solvents containing 0-99% water, the remainder of the solvent being composed of a water-miscible solvent. Normal pH measurements may be made between 99 and 100% water concentrations and are expressed herein as ^{W}_{W}pH wherein the pH measurement is made in water with a pH meter previously calibrated in water. Such a measurement of ^{S}_{W}pH, is made in either the nonaqueous solvent designated by the superscript or in mixtures of up to 99% water in the non-aqueous solvent.

The ammonium compound utilized in the process of the present invention may be selected from one or more ammonium salts of inorganic and organic acids including polymeric ammonium salts such as ammonium salts of polymethacrylic and polyacrylic acid. The ammonium compound also may be selected from ammonia ligands containing metals, e.g., ligands having the formula M(NH₄)m Xⁿ⁻ wherein M is a metal, X is an anion, m is 1 to 10 and n is 1 to 3, either alone or in combination with other ammonium compounds described herein. Typical ammonium salts have the formula (NH₄)ₙ⁺ X^{n -} wherein X is the residue of an acid anion and n is 1, 2 or 3. Examples of the ammonium compounds include ammonium chloride, ammonium bromide, ammonium hydrogen sulfate or diammonium sulfate or mixtures thereof, monobasic ammonium phosphate, dibasic ammonium phosphate or tribasic ammonium phosphate or mixtures thereof, ammonium acetate, ammonium formate, ammonium trifluoroacetate, ammonium oxalate, ammonium salicylate, ammonium citrate, ammonium nitrate and mixtures thereof. A preferred inorganic source of ammonium ion is ammonium chloride. Optionally, a mixture of an ammonium salt and the conjugate acid of one or more ammonium salts may be used as solvent or co-solvent either in solution or in a melt, i.e., a melt of an ammonium salt may function as a solvent or co-solvent, e.g., fructose may be dissolved in molten ammonium acetate/acetic acid in the presence of methanol, or to produce and/or maintain the acidity of the reaction mixture at a particular level. The concentration of the ammonium compound or compounds in the solvent may range from about 1 to 80 weight percent based on the total weight of the reaction mixture. Concentrations of about 1 to 15 weight percent (same basis) are more typical. Preferred ammonium compounds for use as an ammonia source include mixtures of ammonia and ammonium salts to maintain aqueous or nonaqueous pH at a desired value or within a desired range. Such ammonium salts include ammonium salts of carboxylic acids, e.g., carboxylic acids having up to about 6 carbon atoms including trifluoroacetic acid, mono, di or tribasic ammonium phosphate, and mono or diammonium sulfate and mixtures thereof. The process normally is carried out using an ammonium compound or compounds that provide ammonia or ammonium ion in a molar amount equal to or in excess of the molar amount of glucosamine to be produced. The ammonium compound or compounds normally are not used in an excess that affects significantly the purity of the product. The molar amounts of ammonia or ammonium ion employed typically are about 0.5 and 10 moles, more typically about 0.5 to 5 moles, per mole of fructose used. When solubility of the ammonium compound is limited, about 0.5 to 2 moles of ammonia or ammonium ion may be used per mole of fructose.

The process of the present invention is carried out in the presence of a solvent such as water or an inert, organic, water miscible solvent. As used herein to describe the organic, water miscible solvent, ‘inert’ means that the solvent does not incorporate in the product or co-product formed. As used herein, ‘water miscible solvent’ means solvents which are miscible or soluble when mixed or combined with water and have the property of miscibility with water wherein miscibility is the property of mixing or becoming homogeneous, e.g., as miscibility is defined in Webster's Third New International Dictionary Unabridged, 1981. Preferred water-miscible solvents include dimethylsulfoxide, acetonitrile, acetone, tetrahydrofuran, C1-C6 carboxylic acids, e.g., acetic acid, glycols and alcohols, e.g., an alkanol containing 1 to 12 carbon atoms. Preferred alcohols and glycols for use in the process are methanol, ethanol, propanol, 2-propanol, ethylene glycol, 1,2-propanediol, 1,3 propane-diol, n-butanol, isobutanol, and t-butanol. The most preferred solvents are methanol, ethanol, water and mixtures thereof. Methanol is especially preferred due to the insolubility of glucosamine salts such as glucosamine hydrochloride and the relatively high solubility of fructose and of ammonium salts such as ammonium chloride. It will be apparent to those skilled in the art that the use of high fructose corn syrup will result in the process being carried out in the presence of at least some water. Some water will be present during the operation of the process since water is a byproduct of the reaction of fructose with ammonia and ammonium salts. The process of the present invention preferably is carried out in the presence of a solvent comprising an inert, organic, water-miscible solvent containing up to about 10 weight percent, e.g., about 1 to 10 weight percent, water.

In one embodiment of the invention, fructose is contacted or reacted with ammonia, an ammonium compound or a mixture thereof in the presence of a solvent comprising about 25 to 100 weight percent water and 75 to 0 weight percent of an inert, organic, water-miscible solvent at a ^{W}_{W}pH or ^{S}_{W}pH of about 1 to 6. Thus, in this embodiment, the process is carried out under acidic conditions meaning the reaction mixture comprising fructose, an ammonium compound and solvent has a ^{W}_{W}pH or ^{S}_{W}pH of less than 6. This embodiment of the process preferably is carried out in a solvent comprising about 50 to 100 weight percent water and 0 to less than 50 weight percent of an inert, organic, water-miscible solvent contains an inorganic or organic acid that imparts a ^{W}_{W}pH or ^{S}_{W}pH of less than about 6 to the solvent, wherein ^{W}_{W}pH or ^{S}_{W}pH is measured by pH meter.

In another or second embodiment of my novel process, fructose is contacted or reacted with ammonia, an ammonium compound or a mixture thereof in the presence of a solvent comprising about 75 to 100 weight percent of an inert, organic, water-miscible solvent and 0 to 25 weight percent water at a ^{S}_{W}pH of about 1 to 10. Preferably, the process is carried out in an inert, organic, water-miscible solvent containing up to about 10 weight percent water, e.g., about 1 to 10 weight percent water, and a base such as ammonia optionally in combination with an inorganic or organic acid that imparts a ^{S}_{W}pH of less than about 6, preferably about 2 to 6, to the solvent.

The pH of the reaction mixture may decrease during the operation of the process of the present invention. The ^{W}_{W}pH or ^{S}_{W}pH may be controlled continuously, e.g., by addition of acid or base wherein the preferred base is ammonia or in the presence of a buffer system which results in a ^{W}_{W}pH or ^{S}_{W}pH in the desired range. Examples of such buffer systems include amines and amine/acid addition salts such as pyridine/pyridine hydrochloride and imidazole/imidazole hydrochloride and (1) an ammonium salt of an acid and (2) the conjugate acid. Examples of preferred buffer systems include at least one carboxylic acid and the ammonium salts thereof, e.g., ammonium acetate/acetic acid, ammonium formate/formic acid, ammonium citrate/citric acid, ammonium salicylate/salicylic acid, and trifluoroacetic/ammonium trifluoroacetate; sulfuric acid/ammonium hydrogen sulfate or diammonium sulfate or mixtures thereof, phosphoric acid/and mono-, di- or tri-basic ammonium phosphate or mixtures thereof, and partial ammonium salts of aryl or alkyl phosphonic acids such ammonium phenylphosphonate/phenyl phosphonic acid. The most preferred buffer systems are ammonium acetate/acetic acid, ammonium formate/formic acid, ammonium salicylate/salicylic acid, ammonium citrate/citric acid, pyridine/pyridine hydrochloride, imidazole/imidazole hydrochloride and combinations thereof in a concentration that imparts a ^{W}_{W}pH or ^{S}_{W}pH of 1 to 6 to the solvent. Although the use of a buffer system is preferred, a buffer system is not essential. Instead the ^{W}_{W}pH or ^{S}_{W}pH may be controlled by periodic or continuous addition of an acid and/or base to the reaction mixture. Preferred bases to control ^{W}_{W}pH or ^{S}_{W}pH by periodic or continuous additions are ammonia and ammonium salts. A combination of periodic or continuous pH control and a buffer system also may be used. Preferred acids to control pH are inorganic acids such as sulfuric, phosphoric and hydrochloric acid and C1-C6 carboxylic acids.

The process provided by the present invention may be carried out at a temperature of about 0 to 150° C., more typically at a temperature of about 25 to 100° C. and for the preferred lower boiling solvents the temperature more typically is between about 25 and 80° C. Pressure is not an important feature and therefore the process typically is operated at ambient pressure although pressures moderately above or below ambient pressure may be used. Preferred glucosamine acid addition salts include the addition salts of glucosamine with strong mineral acids such as glucosamine hydrochloride, glucosamine sulfate, glucosamine sulfate.2KCl, and glucosamine sulfate.2NaCl.

In a particularly preferred embodiment, ammonium chloride is the inorganic ammonium source and glucosamine hydrochloride is formed as an insoluble precipitate. C1-C3 alcohols are preferred water-miscible solvents with methanol being particularly preferred. When methanol is used in the process as a solvent, preferably in the presence of less than 10 weight percent water, the glucosamine hydrochloride product may be further purified by recrystallization or reslurry in methanol. While many temperatures may be employed in the recrystallization or reslurry of glucosamine hydrochloride in methanol, e.g., 0 to 150° C., it is more preferable to use temperatures between room or ambient temperature (25° C.) and the boiling point of methanol (65° C.). Glucosamine hydrochloride may be separated from mannosamine hydrochloride in the initial precipitation from methanol or in the purification from methanol Furthermore, when glucosamine hydrochloride is precipitated from the production process, the overall ratio in the reaction mixture of glucosamine to mannosamine improves to greater than 4:1, typically to greater than 5:1 and that the mannosamine content of the isolated or purified solids is under 2 weight percent. A further advantage of this precipitation or crystallization of glucosamine from the reaction mixture is that relatively high concentrations of mannosamine can be obtained from the filtrate, i.e., mannosamine remains in solution. Mannosamine is useful as an intermediate in the synthesis of specialty and fine chemicals. Mannosamine preferably is obtained as an acid addition salt selected from those acid addition salts which are preferred for glucosamine. A highly preferred acid addition salt for mannosamine is mannosamine hydrochloride. The process may be operated in a continuous, semi-continuous or batch mode of operation. Semi-continuous or continuous operation has the advantage that the glucosamine hydrochloride may be removed as it precipitates, thereby minimizing any decomposition.

It is advantageous to select or control the concentration of reactant materials, e.g., the fructose and ammonia source such as ammonium chloride, in the reaction solvent such that an acid addition salt of glucosamine is produced from fructose and the ammonium source at a concentration above its solubility limit, thereby causing precipitation or crystallization of at least some and preferably most of the glucosamine salt as it is formed. Crystallization or precipitation of the acid addition salt of glucosamine as it is formed minimizing decomposition of the glucosamine salt. The solvent may comprise only water, only an inert, water-miscible solvent or a mixture thereof wherein the solvent further reduces the solubility of glucosamine acid addition salts. Examples of such solvents are described above. When mixtures of water and an inert, water-immiscible solvent are used, the initial solids concentration either dissolved or as a slurry in the reaction solvent typically is above 10 weight percent based on the total weight of the reaction mixture. It is apparent that the solubility of the reactants and glucosamine acid addition salts will vary depending upon the particular solvent and materials employed. Preferred glucosamine salt forms are those of the strong acids, e.g., glucosamine sulfate, glucosamine hydrochloride and glucosamine phosphate. Operation of the process under conditions that result in the precipitation or crystallization of the glucosamine acid addition salt requires utilization of relatively large amounts or concentrations of fructose and ammonium compound. Typical total amounts of fructose utilized in the process in either batch, continuous or semi-continuous mode of operation exceeds 1 weight percent of the total reaction mixture. More typically, total amounts of fructose fed to the reaction in either batch, continuous or semi-continuous mode, is greater than 5 weight percent, preferably greater than 15 weight percent, of the total reaction mixture.

### EXAMPLES

The process of the present invention is further illustrated by the following examples wherein all percentages are by weight unless otherwise specified. Unless noted otherwise, all reactions were stirred magnetically. Water used was either HPLC grade or filtered through a Millipore ion exchange system. Except where stated otherwise, fructose is 99% crystalline D-fructose purchased from Aldrich (Catalog No. 239704-50g). D-Glucosamine hydrochloride and D-mannosamine hydrochloride standards were purchased from Sigma-Aldrich and were at least 98% pure according to the manufacturer. Ortho-pthaladehyde reagent (Product No. 26025; containing 0.8 mg/ml of o-pthaldehyde, Brij-35, mercaptoethanol in a borate buffer) was purchased from Pierce Chemical Company. Methanol used for HPLC was HPLC grade solvent. 20 Millimolar pH 7 phosphate buffer was prepared using 10 millimoles (mmol) of mono-potassium phosphate and 10 millimoles of di-potassium phosphate per liter of water.

HPLC results reported are molar percent conversions (yield) or molar percent remaining. These results are thus independent of the salt form of the glucosamine. Thus as reported for these HPLC methods, results may interchangeably refer to either glucosamine hydrochloride (used always as standards) or glucosamine free base. As used herein, yield refers to moles of product formed/moles of starting material. Where more than one product form is obtained, e.g., solids and filtrate, yield may be either summed between the two product forms or expressed for each product form, e.g. yield in solids and yield in filtrate.

The HPLC fluorescence method (FLD method—pre-column derivitization) was a modification of the method of Dominquez, et. al., Journal of Chromato-graphic Science, 25, 468 (1987). All HPLC analyses were run on an Agilent 1100 with autosampler and analyzed using Agilent ChemStation Software (2004). Fluorescence detection utilized an HP 1046A Programmable Fluorescence Detector (220 nm excitation wavelength, 455 nm Emission detection and a gain setting of 10). High pressure chromatography was carried out using an Agilent Zorbax SB-C 18 column (3.5 um packing, 4.6×7.5 mm, Part No. 866953-902) at a flow rate of 1.8 ml/minute using isocratic conditions of 20% methanol and 80% 20 millimolar phosphate buffer, pH 7, for the first 5.5 minutes and then gradient elution (flow at 1.5 ml/minute) for the next 8 minutes to a final solvent composition of 50% methanol/50% aqueous buffer. Precolumn derivitization was accomplished using the autosampler as follows: At room temperature, 2 microliters (μl) were drawn from the sample solution, 10 μl were drawn from the Pierce o-pthaldehyde reagent solution (Pierce Fluoraldehyde product 26025) and the entire 12 μl was mixed in the seat three times. The injector was programmed to wait 2 minutes after mixing. This online derivitization procedure was used due to the reported instability of the formed isoindole of glucosamine by Dominquez and others. Glucosamine and mannosamine were quantified in unknown solutions (after dilution). The external standard determination of glucosamine has been found to be linear between 1 ppm and 50 ppm for glucosamine and linear between 5ppm and 50 ppm for mannosamine when ammonium levels of the sample were under 6 millimolal when calibrated on a 25 ppm standard of glucosamine hydrochloride and mannosamine hydrochloride (single point calibration). Relative standard deviations for standard mixtures of glucosamine hydrochloride and mannosamine hydrochloride were approximately 10%-40%. Results reported for the FLD methods are the average of 3 measurements of the same sample. Diluted samples were either stored at room temperature and analyzed within the working day or stored at −80° C. before analysis.

An HPLC based pulsed amperiometric detection method was developed for glucosamine, mannosamine, glucose and fructose using a Dionex Carbopac PA10 column with 20 millimolar sodium hydroxide as eluent based on the method of Fosdick et. al., US 2004/0077055 A1, Example 3. Relative standard deviations for standard mixtures and for experimental samples were typically under 10% (combined sampling and analytical error) for glucosamine hydrochloride and fructose using this HPLC based pulsed amperiometric detection method. Unless otherwise specified, pH meters were calibrated using standard buffers (at pH 4, 7, 10) in water at room temperature. Measurements of ^{W}_{W}PH or ^{S}_{W}pH were then made at the indicated temperature and solvent.

### Example 1

This example demonstrates the production of glucosamine from fructose and ammonium chloride in methanol containing an ammonium acetate/acetic acid buffer system. Ammonium chloride (5.5 g, 0.103 moles), ammonium acetate (1.83 g, 23.7 mmol) and acetic acid (1.41 g, 23.5 mmol) were dissolved in anhydrous methanol (392.4 g). The pH of the resulting buffer solution measured on water wet narrow range pH paper was approximately 5.5. The calculated molality of ammonium ion in this buffer (ammonium acetate and ammonium chloride) was 0.32 molal.

Fructose (1.44 g, 8 mmol) and the buffer solution described in the preceding paragraph (150 ml, 120.9 g, 39 mmol ammonium) were added under an inert atmosphere of argon and heated at 55° C. for 2 days. The pH determined by spotting the reaction mixture on water-wet, narrow range pH paper was approx 5.3 at the start of reaction (measured at 4.5 at one and two days). Aliquots were removed periodically for HPLC analysis. All reactants and products remained in solution throughout the course of the reaction. HPLC analysis (FLD method) gave the following conversions (single pass yield): 6 hours, 14% glucosamine; 25 hours, 20% glucosamine. HPLC analysis (PAD) method): 25 hours, 18% glucosamine; 4.6% mannosamine (glucosamine:mannosamine ratio ca. 4/1; 12% remaining fructose.

### Example 2

This example is a larger scale version of Example 1 and demonstrates the purification of the glucosamine product. Ammonium chloride (27.89 g, 0.521 moles), ammonium acetate (9.15 g, 0.119 moles), acetic acid (7.8 g, 0.130 moles), fructose (24.12 g, 0.134 moles) and methanol (1967.8 g) were charged to a 3 L flask under argon. The initial pH of the resulting solution was measured at 5.5 on water wet pH paper. The reaction solution was heated to 60° C. and the course of the reaction was monitored by periodic removal of aliquots and analysis by HPLC. A solution was maintained throughout the course of the reaction. After approximately 28.5 hours the solution pH had dropped to 4.4 and heating was discontinued. Concentrated hydrochloric acid was added to a pH of approximately 2.5 on water-wet, narrow range pH paper. This pH-adjusted solution was allowed to stir at room temperature overnight. HPLC data prior to neutralization (FLD method, single pass yield): approx 5 hours, 18% glucosamine (glucosamine:mannosamine ratio approximately 4:1); approximately 23.5 hours, 28% glucosamine; approximately 28.5 hours (immediately prior to acidification), 31% glucosamine (glucosamine:mannosamine ratio approximately 7:1).

The reaction solution was concentrated on a rotary evaporator (bath temperature of 26° C.) to a volume of less than 500 ml. Filtration produced a filtrate which was dried by evaporation of solvent to a constant weight to provide 27.77 g of an oil and two solids of apparently different densities which partially separated on pouring into the filter and suction drying. These two solids were partially separated with a spatula into an upper layer (15.73 g) and a lower layer (15.78 g). These solids were dried under high vacuum (<1 Torr) to constant weight. HPLC analysis (FLD method) of the two solids provided the following weight percent compositions; upper layer of solids: 25% glucosamine hydrochloride; lower layer of solids: 5.7% glucosamine hydrochloride; oil: 6.5% glucosamine hydrochloride and 4% mannosamine hydrochloride. HPLC analysis (PAD method) provided the following weight percent compositions; upper layer of solids 27.6% glucosamine hydrochloride and 0.59% mannosamine hydrochloride; lower layer of solids 6.5% glucosamine hydrochloride and 0.26% mannosamine hydrochloride; oil: 8.1% glucosamine hydrochloride, 4.7% mannosamine hydrochloride and 4.8% fructose (glucose was not detected). The bulk of the remainder is assumed to be ammonium salts.

Based on the results obtained from the two HPLC methods, isolated yields may be calculated based on the assays of isolated materials. Thus, by the FLD method there was approximately 3.93 g of glucosamine hydrochloride in the lower density upper layer (18.2 mmol, 13.6% yield), approximately 0.90 g of glucosamine hydrochloride in the lower layer (4.2 millimoles, 3.1% yield), and approximately 1.80 g of glucosamine hydrochloride in the oil (8.4 millimoles, 6.3% yield). The combined yield based on analysis by the FLD method of isolated glucosamine hydrochloride was 30.8 millimoles or 23% based on initial moles of fructose. Comparatively, from the PAD method there was approximately 4.34 g of glucosamine hydrochloride in the lower density upper layer (20.1 millimoles, 15% yield), approximately 1.03 g of glucosamine hydrochloride in the lower layer of solids (4.8 mmol, 3.6% yield), and approximately 2.25 g of glucosamine hydrochloride in the remaining oil (10.4 mmol, 7.8%). The combined yield based on analysis by the PAD method of isolated glucosamine hydrochloride was 35.3 millimoles or 26% based on initial moles of fructose.

### Example 3

This example demonstrates the production of glucosamine from fructose and ammonium bromide in an ammonium acetate/acetic acid buffered methanol solution. A stock solution of ammonium bromide (43 g, 0.44 moles), ammonium acetate (1.82 g, 23.6 millimoles) and acetic acid (1.41 g, 23.5 millimoles) was prepared in anhydrous methanol (390.59 g) under an argon atmosphere. The calculated molality of ammonium ion in the buffer (ammonium acetate and ammonium bromide) was 1.06 molal (moles/kg solution). Fructose (1.43 g, 8.0 millimoles) and the methanol buffer solution of ammonium bromide described herein (150 ml, 125.9 g, 133 millimoles ammonium ion) were charged to a thermowell-equipped flask under an inert atmosphere of argon and heated to 55° C. for 30 hours. The initial pH of the solution was 5.5 at the start of reaction and at 4.7 after 24 hours when measured on water-wetted, narrow range pH paper. HPLC analysis (FLD method) gave the following conversions (single pass yield): 5.5 hours, 17% glucosamine; 24 hours, 33% glucosamine, 6.46% mannosamine (glucosamine/mannosamine ratio=5:1); 30 hours, 36% glucosamine. HPLC analysis (PAD) method): 24 hours: 29% glucosamine, 7.6% mannosamine (glucosamine:mannosamine ratio=4:1); 12% unreacted fructose.

### Example 4

This example demonstrates the production of glucosamine under conditions in which it precipitates. Ammonium chloride (27.89 g, 0.521 moles), ammonium acetate (9.13 g, 0.118 moles), acetic acid (7.33 g, 0.122 moles) and methanol (195.96 g) were charged to a 500 ml flask equipped with a mechanical stirrer and thermowell. To this stirred suspension of solids was added 98% crystalline fructose (24.53 g, 0.136 moles). The stirred reaction mixture containing suspended solids was heated overnight at 60° C. The following morning, the reaction mixture which still contained significant suspended solids was allowed to cool and the pH was adjusted by addition of concentrated aqueous hydrochloric acid until the solution pH was 2.5 to 3 as measured by application of the solution onto water-wet pH paper. The resulting solids were collected by filtration on sintered glass funnel and dried by air suction (Precipitate 1, 29.91 g). The resulting filtrate was partially evaporated and a second filter cake was obtained by filtration and suction drying (Precipitate 2, 10.81 g). The resulting filtrate was concentrated to a viscous oil first on a rotary evaporator and finally by vacuum drying (<1 Torr) on a freeze dryer (oil, 20.28 g).

HPLC results (PAD method) gave the following assays: Precipitate 1 (23.1% glucosamine hydrochloride, no detectable mannosamine or fructose, yield glucosamine hydrochloride in Precipitate 1=24%), Precipitate 2 (0.69% glucosamine hydrochloride, 0.13% mannosamine hydrochloride), oil (7.0% glucosamine hydrochloride, 0.51% mannosamine hydrochloride, yield of glucosamine hydrochloride in this oil=4.8%). HPLC results (FLD method) gave the following assay: 23.3% glucosamine hydrochloride, yield of glucosamine hydrochloride in Precipitate 1=24%). The bulk of the remainder is assumed to be ammonium salts.

### Example 5

This example is a replication of Example 4 with a shortened reaction time. Ammonium chloride (27.69 g, 0.520 moles), ammonium acetate (9.49 g, 123 mmol), acetic acid (7.26 g, 121 mmol) and methanol (192.5 g) were charged to a 500 ml flask equipped with a mechanical stirrer and thermowell. To this mixture was added 98% crystalline fructose (24.14 g, 134 millimoles). This reaction mixture was stirred at approximately 55° C. for approximately 5 hours. At no point did all of the solids go into solution. (In a similar experiment using a pH meter, ^{S}_{W}pH was measured at 5.2 upon the reaction mixture reaching 55° C. and was a ^{S}_{W}pH of 4.7 after 4 hours at 55° C. wherein s=methanol and w=water). This stirred mixture then was placed in a room temperature water bath and upon cooling to near room temperature, the pH was adjusted from approximately 4.5 to approximately 2 by addition of concentrated aqueous HCl (pH as measured by application of the solution onto water-wet, pH paper) to cause formation of a further precipitate. The resulting mixture was immediately filtered and the solids collected were air and vacuum dried (overnight at approximately less than 1 Torr) to provide 25.38 g of solids (Precipitate 1) which were ground with a mortar and pestle to ensure homogeneity. The resulting filtrate was partially concentrated in vacuum to produce a second precipitate and again filtered to obtain 8.23 g of solids (Precipitate 2) which was presumed to be ammonium chloride as it was later shown to have a negligible amounts of glucosamine and fructose (less than 1%). The resulting filtrate was dried to approximately constant weight on a rotary evaporator and under high vacuum overnight to provide an oil (21.6 g).

HPLC (PAD method) results gave the following assay: Precipitate 1 (42.5% glucosamine hydrochloride, 0.53% fructose, 0.29% mannosamine hydrochloride; yield of glucosamine hydrochloride=37%); oil (6.6% glucosamine hydrochloride, 2.4% mannosamine hydrochloride, 8.1% fructose). NMR (using DMSO as an internal standard): Precipitate 1 (44% glucosamine hydrochloride, 0.28% fructose; yield of glucosamine hydrochloride in Precipitate 1=39%); oil (6.8% glucosamine hydrochloride, 3.4% mannosamine hydrochloride, 5.4% fructose).

### Example 6

This example demonstrates the production of glucosamine in an initial purity of greater than 50% and using lesser amounts of ammonium chloride and methanol to facilitate production. A three-neck, glass reaction vessel equipped with a thermowell was charged with ammonium chloride (7.29 g, 136 mmol), ammonium acetate (9.27 g, 120 mmol), acetic acid (7.13 g, 119 millimoles) and methanol (100.66 g). To this magnetically stirred mixture was added 98% crystalline fructose (24.03 g, 133 mmol) and the stirred mixture was heated at 55° C. for approximately 5 hours. Upon reaching 55° C., nearly all of the solids dissolved. After approximately 30 minutes a precipitation or crystallization was observed to start and the amount of precipitated solids increased over time. After the reaction mixture had been stirred for 5 hours at 55° C., the reaction was cooled to room temperature using a water bath and was partially neutralized by addition of concentrated HCl (6.05 g, approximately 61 millimoles) to a pH of approximately 2 (measured by application of the reaction solution onto water wet narrow range pH paper). The mixture was allowed to stir overnight at room temperature and was then filtered. The collected solids were air dried (approximately 30 minutes) by suction and was then vacuum dried (approximately less than 1 Torr) to provide 8.27 g of solids. The resulting filtrate was dried to approximately constant weight on a rotary evaporator and under high vacuum overnight to provide and oil (23.01 g).

HPLC (PAD method) results gave the following assay: solids (70% glucosamine-measured as hydrochloride against calibrated standard, 0.44% fructose, 0.49% mannosamine, measured as hydrochloride; yield of glucosamine hydrochloride in solids=20%); oil (3.7% glucosamine-measured as hydrochloride, 1.6% mannosamine—measured as hydrochloride, 5.2% fructose). NMR (using DMSO as an internal standard): solids (75% glucosamine—expressed as percent hydrochloride, yield of glucosamine hydrochloride in solids=22%), oil (1.68% glucosamine hydrochloride, 2.57% mannosamine hydrochloride, 4.43% fructose).

### Example 7

This example demonstrates the production of glucosamine using a buffer of ammonium formate and formic acid in methanol with ammonium chloride. A three-neck, glass reaction vessel equipped with a thermowell was charged with ammonium chloride (7.28 g, 136 mmol), ammonium formate (7.56 g, 120 mmol), methanol (98.32 g) and formic acid (5.53 g, 120 mmol) under an inert atmosphere of argon. To the magnetically stirred mixture was added 98% crystalline fructose (24.01 g, 133 millimoles) and the reaction mixture was heated to 55° C. for approximately 6 hours. Nearly all of the solids initially went into solution. Copious precipitation of solids or crystals occurred after approximately one hour at 55° C. (In a similar experiment using a pH meter, ^{S}_{W}pH was measured at 4.1 upon the reaction mixture reaching 55° C. and was also a ^{S}_{W}pH of 4.1 after 4 hours at 55° C. wherein s=methanol and w=water). After heating for approximately 6 hours, the reaction mixture was cooled to room temperature using a room temperature water bath and then partially neutralized with concentrated aqueous HCl (8.62 g, approximately 87 millimoles) to a pH of approximately 2 as measured by water-wet, narrow range pH paper and left to stir at room temperature overnight and was then filtered. The collected solids were air dried for approximately 30 minutes by suction and then vacuum dried (approximately less than 1 Torr, overnight) to provide 9.4 g of solids. The resulting filtrate was dried to approximately constant weight by rotary evaporation and then vacuum dried (approximately less than 1 Torr, 5 hours).

HPLC (PAD method) results gave the following assay: solids (64% glucosamine hydrochloride, 2.3% fructose, 0.8% mannosamine hydrochloride; yield of glucosamine hydrochloride in solids=21%). NMR (using DMSO as an internal standard); solids (66% glucosamine hydrochloride; yield of glucosamine hydrochloride in solids=22%).

### Example 8

This example demonstrates the purification of glucosamine hydrochloride by crystallization from water. The first isolated solids from Example 2 (5.04 g) were added to water (10.05 g) at 60° C. A lightly colored solution was obtained. This solution was stored at 5° C. for 11 days. The formed crystals (0.669 g) were isolated by filtration and vacuum dried. The crystals:assayed 92% glucosamine hydrochloride by HPLC (PAD method) and 86% glucosamine hydrochloride by Proton NMR (DMSO internal standard). Ammonium analysis provided an assay of 2.0% ammonium ion which is consistent with the presence of approximately 6% ammonium chloride.

### Example 9

This example demonstrates the production of glucosamine from fructose and ammonium chloride using ammonium acetate buffer in water at a concentration that results in the precipitation of the glucosamine formed. A 500 ml 3-neck, round-bottomed flask equipped with a mechanical stirrer and a thermowell was charged with ammonium chloride (43.86 g, 0.820 moles), ammonium acetate (55.53 g, 0.717 moles), distilled water (49 g) and glacial acetic acid (42.88 g, 0.714 moles) under an inert atmosphere of argon. To this stirred slurry of solids was added fructose (145.04 g, 0.855 moles) and the reaction mixture was heated to 55° C. The liquid quickly became too dark to observe the nature of the solids in solution. After heating for a total of approximately 4 hours, the reaction was cooled to room temperature by application of an external room temperature water bath. The stirred mixture was then partially neutralized by addition of concentrated aqueous HCl (61.29 g, approximately 0.618 moles, to a pH of approximately 1.5 to 2) and the mixture was allowed to stir overnight at room temperature. On the following morning, the resulting solids were collected, suction dried and dried under high vacuum (<1 Torr) overnight to provide 46.23 g of crude solid product. The filtrate from the filtration was dried for several days on a freeze dryer. Proton NMR (DMSO internal standard) gave the following assay on the solids: 24.49% glucosamine hydrochloride (corresponding to a yield of glucosamine in the crude solids of approximately 7%).

### COMPARATIVE EXAMPLE 1

This example demonstrates the production of glucosamine from fructose and ammonia in methanol under highly basic conditions outside the scope of the present invention. Fructose (10.01 g, 55.6 mmol) and 7.39 molar ammonia in methanol (150 ml, 116.94 g, 1.11 moles NH₃) were charged to a flask under argon at room temperature (24° C.). The flask was stoppered to prevent evaporation of ammonia vapor. At the start of the reaction and after one day the ^{S}_{W}pH (s=methanol) was measured at 12.8. The reaction was allowed to stir for 6 days at room temperature with periodic removal of aliquots for HPLC analysis. The calculated initial molality of ammonia was 8.7 molal (moles/kg solution). HPLC analysis (FLD method) gave the following conversions (single pass yield): 24 hours, 1% glucosamine, 45 hours 1.6% glucosamine, 69 hours 4.1% glucosamine (glucosamine:mannosamine ratio=7:1), 144 hours 5.2% glucosamine (glucosamine:mannosamine ratio=10:1). HPLC analysis (PAD method): 69 hours, 2.4% glucosamine (glucosamine:mannosamine ratio 4:1); 144 hours, 4.1% glucosamine (glucosamine:mannosamine ratio=5:1).

### COMPARATIVE EXAMPLE 2

This example demonstrates the production of glucosamine from fructose in liquid ammonia and is similar to Example 1 of U.S. Pat. No. 2,884,411 except that the reaction was stopped at 2 hours instead of 6. Fructose (2.52 g, 14 mmol) was charged to a 300 ml autoclave constructed of Hastaloy C alloy which was purged with dry nitrogen. Anhydrous liquid ammonia (100 ml) was added via blowcase. The reaction was sealed and heated at 100° C. for 2 hours with stirring. The reactor was air cooled to 25-35° C. and then carefully vented. The reactor was then opened and contents dissolved in two portions (washes of 150 ml each) of 3% aqueous formic acid. The two portions (washes) were combined (pH=3.9) and freeze dried (4.99g). HPLC analysis of the freeze dried solids (FLD method) gave the following conversion (single pass yield): 1.8% glucosamine.

### COMPARATIVE EXAMPLE 3

This example demonstrates the production glucosamine from fructose and concentrated ammonium hydroxide and is similar to Example 3 of U.S. Pat. No . 2,884,411 with the notable exception that in Example 3 of U.S. Pat. No. 2,884,411 the reaction was carried out at 100° C. for 2 hours while the present example monitors the reaction at room temperature. Fructose (10.99 g, 61 mmol) and concentrated 29% aqueous ammonium hydroxide (103.6 g, 1.8 moles of ammonia) were added to a 250 ml flask under a nitrogen atmosphere at room temperature and degassed with nitrogen for approximately 15 minutes (subsurface addition via 18 gauge syringe needle, any liquid removed was recondensed and returned the flask (approximately 2 drops) by use of a −78° C. condenser). The reaction mixture was stoppered to prevent loss of ammonia vapor and stirred at room temperature for 11 days. Aliquots were periodically removed and monitored by HPLC. The pH as measured by application of an aliquot onto wetted narrow range pH paper was approximately 12 throughout the reaction.

HPLC analysis (FLD method) gave the following conversions (single pass yield): 19 hours, approximately 0.4% glucosamine; 26 hours, approximately 0.6% glucosamine; 48 hours, approximately 1.1% glucosamine; 69 hours, approximately 1.9% glucosamine; 262 hours, 3.8% glucosamine (approximate ratio of glucosamine/mannosamine=5/1) HPLC analysis (PAD) method): 262 hours, 3.3% glucosamine (approximate glucosamine:mannosamine ratio=10:1), approximately 6.5% of the fructose remained (PAD) and 5.8% glucose was formed (single pass yield).

### COMPARATIVE EXAMPLE 4

This example demonstrates the production of glucosamine from fructose using an ammonium bicarbonate buffer in water. Fructose (1.85 g, 10.3 mmol), ammonium hydrogen carbonate (4 g, 50.6 mmol) and water (100.55 g) were charged to a 250 ml flask under argon. The reaction mixture was allowed to stir at room temperature (24° C.) and monitored periodically by removal of an aliquot (ca. 1 ml). The pH was found to be approximately 8-9 upon testing with wide range pH paper. HPLC analysis (FLD method) gave the following conversions (single pass yield): 4 days, 0.17% glucosamine; 7 days 0.3% glucosamine, 11 days 0.6% glucosamine. Mannosamine also was detected in trace amounts.

### Example 10

This example demonstrates the purification of glucosamine hydrochloride by reslurry from methanol. Example 6 was repeated at 10× scale to provide an initial precipitate/crystals from the reaction of 87.73 g of solids (yield of glucosamine hydrochloride in these solids was approximately 19%, purity of glucosamine hydrochloride was 61.9% in these solids by PAD/HPLC, also present were 1.11% fructose and 0.67% mannosamine hydrochloride (PAD); ion chromatography found 8.2% ammonium as NH₄ which corresponds to a calculated ammonium chloride content of 24.3%.). A portion of these solids (43.79 g, estimated to contain 27.1 g of glucosamine hydrochloride from PAD/HPLC assay) and methanol (500 ml) were added to a glass reaction vessel and brought to reflux for 5 minutes with mechanical stirring. While hot, this mixture was poured through a coarse fritted sintered glass filter funnel. The resulting solids were air dried and then placed on a freeze dryer until at constant weight (25.85g). The resulting filtrate was concentrated in vacuuo and then dried on the freeze dryer to a gummy solid (16.32 g, referred to in the assay below as an oil).

Solids assay from reslurry experiment: 94.8% glucosamine hydrochloride, 0.24% mannosamine hydrochloride, 0.1% fructose (PAD); ion chromatography ammonium assay, 0.63% as NH₄ which corresponds to 1.87% ammonium chloride when adjusted for molecular weights of NH₄ (18.04) and ammonium chloride (53.49), ion chromatography chloride assay, 17.1% (expected=16.8% chloride in a composition of 94.8% glucosamine hydrochloride and 1.87 weight % ammonium chloride). Oil assay: 8.0% glucosamine hydrochloride.

### Example 11

This example demonstrates the use of ammonium salicylate/salicylic acid as a buffer in methanol and the isolation of glucosamine without a hydrochloric acid neutralization. A solution of 7.44 N ammonia in methanol (16.7 ml, 124 mmol) was added to methanol (98.4 g, 125 ml) and salicylic acid (248 mmol) in a 3-neck reaction flask equipped with thermowell and magnetic stirrer. Ammonium chloride (8.5g, 159 mmol) and fructose (24.52 g, 136 mmol) were added and the reaction mixture was heated to 55° C. Upon reaching 55° C., the ^{s}_{w}pH was measured at 4.2 (s=methanol) and the reaction was heated for 5 hours and then cooled with a room temperature water bath to room temperature for approximately 1.5 hours. The resulting suspended solids were then filtered through a coarse fritted funnel and suction dried to constant weight (11.85 g). The filtrate (120g) was diluted with a known weight of water and frozen prior to analysis by PAD/HPLC. The combined yield of glucosamine from the filtrate (3.2%) and solids (24.8%) was 28%.

Solids assay: (PAD method): 61.3% glucosamine hydrochloride, 0.89% mannosamine hydrochloride, 2.2% fructose. Undiluted Filtrate assay: (PAD method): 0.79% glucosamine hydrochloride, 1.61% mannosamine hydrochloride, 5.17% fructose (25.3% recovery of fructose in the filtrate).

The invention has been described in detail with particular reference to preferred embodiments thereof, but it will be understood that variations and modifications can be effected within the spirit and scope of the invention.

I claim:

1. Process for the preparation of a glucosamine acid addition salt which comprises contacting fructose with ammonia, an ammonium compound or a mixture thereof in the presence of:
(i) a solvent comprising about 25 to 100 weight percent water and 75 to 0 weight percent of an inert, organic, water-miscible solvent at a ^{W}_{W}pH or ^{S}_{W}pH of about 1 to 6; or
(ii) a solvent comprising about 75 to 100 weight percent of an inert, organic, water-miscible solvent and 0 to 25 weight percent water at a ^{S}_{W}pH of about 1 to 10, wherein ^{S}_{W}pH is measured by a pH electrode in the inert solvent said pH electrode previously being calibrated in water.

2. Process according to claim 1 wherein fructose is contacted at a temperature of about 0 to 150° C. with ammonia, an ammonium compound or a mixture thereof in the presence of a solvent comprising about 75 to 100 weight percent of an inert, organic, water-miscible solvent and 0 to 25 weight percent water wherein the inert, organic, water-miscible solvent is selected from methanol, ethanol, ethylene glycol, 1,2 propane diol, 1,3 propane diol, propanol, 2-propanol, n-butanol, isobutanol, t-butanol and mixtures thereof at ^{S}_{W}pH of less than about 10 to the inert solvent.

3. Process according to claim 1 wherein fructose is contacted at a temperature of about 25 to 100° C. with an ammonium compound selected from ammonium chloride, ammonium bromide, ammonium acetate, ammonium formate, ammonium salicylate, ammonium nitrate, ammonium trifluoroacetate, diammonium phosphate, monoammonium phosphate, diammonium sulfate, and monoammonium sulfate and mixtures thereof in the presence of a solvent comprising about 75 to 100 weight percent of an inert, organic, water-miscible solvent selected from methanol, ethanol, propanol, 2-propanol, n-butanol, isobutanol, t-butanol and mixtures thereof and 0 to 25 weight percent water and a buffer system comprising a combination of an inorganic or organic acid and an ammonium salt thereof in a concentration that imparts a ^{S}_{W}pH of about 1 to 6 to the solvent.

4. Process according to claim 3 wherein the ammonium compound is ammonium chloride, ammonium bromide, ammonium acetate, ammonium formate, ammonium salicylate or a mixture thereof, the solvent comprises an inert, organic, water-miscible solvent selected from methanol, ethanol and mixtures thereof containing up to about 10 weight percent water and the buffer system is a combination of at least one carboxylic acid and the ammonium salt thereof, phenylphosphonic acid and ammonium phenylphosphonate, sulfuric acid/diammonium sulfate, diammonium sulfate/monoammonium sulfate, diammonium phosphate/monoammonium phosphate, phosphoric acid/mono-di or tri-ammonium phosphate, imidazole/imidazole hydrochloride, pyridine/pyridine hydrochloride and mixtures thereof in a concentration that imparts a ^{S}_{W}pH of about 1 to 6 to the solvent and the process is carried out at a temperature of about 25 to 80° C.

5. Process according to claim 3 wherein the ^{S}_{W}pH of the solvent is maintained at about 1 to 6 by addition of ammonia or a combination of ammonia and an inorganic or organic acid.

6. Process according to claim 3 wherein the ammonium compound is ammonium chloride, ammonium bromide, ammonium acetate, ammonium formate or a mixture thereof, the solvent comprises an inert, organic, water-miscible solvent selected from methanol, ethanol and mixtures thereof containing up to about 10 weight percent water and the buffer system is a combination of ammonium acetate/acetic acid, ammonium formate/formic acid, ammonium citrate/citric acid or ammonium salicylate/salicylic acid while maintaining the ^{S}_{W}pH of the solvent in the range of about 2 to 6 and the process is carried out at a temperature of about 25 to 65° C.

7. Process according to claim 6 wherein the solvent comprises methanol containing up to 10 weight percent water.

8. Process according to claim 1 wherein fructose is contacted at a temperature of about 0 to 150° C. with ammonia, an ammonium compound or a mixture thereof in the presence of a solvent comprising about 75 to 100 weight percent of an inert, organic, water-miscible solvent selected from, methanol, ethanol, propanol, 2-propanol, n-butanol, isobutanol, t-butanol and mixtures thereof and 0 to 25 weight percent water at ^{S}_{W}pH of about 1 to 10 wherein ^{S}_{W}pH is controlled by addition of ammonia or a combination of ammonia and an inorganic or organic acid.

9. Process of claim 1 wherein the glucosamine acid addition salt is glucosamine hydrochloride.

10. Process of claim 1 wherein the glucosamine acid addition salt is glucosamine hydrochloride, the solvent comprises methanol containing up to 10 weight percent water and the produced glucosamine hydrochloride is purified by crystallization or reslurrying in methanol containing up to 10 weight percent water.

11. Process according to claim 10 wherein the total amount of fructose fed to the process operated in a batch, continuous or semi-continuous mode is greater than 5 weight percent of the reaction mixture.

12. Process according to claim 10 wherein the total amount of fructose fed to the process operated in a batch, continuous or semi-continuous mode is greater than 15 weight percent of the reaction mixture.

13. Process according to claim 10 wherein the total amount of fructose fed to the process operated in a batch, continuous or semi-continuous mode is greater than 15 weight percent of the reaction mixture and the total amount of ammonia or ammonia ion fed to the process is between 0.5 and 5 equivalents per mole of fructose fed.

14. Process according to claim 1 wherein the glucosamine acid addition salt is glucosamine sulfate or glucosamine sulfate.2KCl.

15. Process according to claim 1 wherein the glucosamine hydrochloride precipitates under the production conditions.

16. Process according to claim 1 wherein a mannosamine acid addition salt is co-produced.

17. Process according to claim 15 wherein the glucosamine hydrochloride precipitates under the production conditions and wherein mannosamine hydrochloride constitutes less than 2 weight percent of the precipitated solids.

18. Process according to claim 1 wherein fructose is contacted at a temperature of about 25 to 150° C. with an ammonium compound selected from ammonium chloride, ammonium bromide, ammonium acetate, ammonium formate, ammonium salicylate, ammonium trifluoroacetate, diammonium phosphate, monoammonium phosphate, diammonium sulfate, and monoammonium sulfate and mixtures thereof in the presence of a solvent comprising about 0 to 25 weight percent of an inert, organic, water-miscible solvent selected from methanol, ethanol, propanol, 2-propanol, n-butanol, isobutanol, t-butanol and mixtures thereof and 75 to 100 weight percent water and a buffer system comprising a combination of an inorganic or organic acid and an ammonium salt thereof or an amine and an amine acid addition salt or a combination thereof in a concentration that imparts a ^{W}_{W}pH or ^{S}_{W}pH of about 1 to 6 to the solvent.

19. Process according to claim 1 wherein fructose is contacted at a temperature of about 0 to 150° C. with ammonia, an ammonium compound or a mixture thereof in the presence of a solvent comprising about 25 to 0 weight percent of an inert, organic, water-miscible solvent selected from, methanol, ethanol, propanol, 2-propanol, n-butanol, isobutanol, t-butanol and mixtures thereof and 25 to 100 weight percent water at ^{W}_{W}pH or ^{S}_{W}pH of about 1 to 6 wherein the ^{W}_{W}pH or ^{S}_{W}pH of the solvent is maintained at about 1 to 6 by addition of ammonia or a combination of ammonia and an inorganic or organic acid.

20. Process according to claim 1 for the preparation of glucosamine hydrochloride which comprises contacting fructose at a temperature of about 25 to 65° C. with ammonium chloride in the presence of a solvent comprising a C1 to C3 alkanol and up to about 10 weight percent water at a ^{W}_{W}pH or ^{S}_{W}pH of about 2 to 6 wherein the ^{W}_{W}pH or ^{S}_{W}pH of the solvent is maintained at about 2 to 6 by addition of ammonia or a combination of ammonia and an inorganic or organic acid, the total amount of fructose fed to the process operated in a batch, continuous or semi-continuous mode is greater than 15 weight percent of the reaction mixture, the total amount of ammonium ion fed to the process is between 0.5 and 5 equivalents per mole of fructose fed, and glucosamine hydrochloride product precipitates from the reaction mixture.
